(19) United States
(12) Patent Application Publication
(10) Pub. No.: US 2012/0003724 A1
(43) Pub. Date: Jan. 5, 2012

(54) Process and Reactor System for Depolymerization of Polymeric Biomass

(75) Inventors: K Ramya, Hyedrabad (IN); Prabhat Goel, Hyedrabad (IN); Kumar Manoj Sarkar, Hyedrabad (IN); Banibrata Pandey, Hyedrabad (IN); Deepti Agrawal, Hyedrabad (IN); Soumya Sasmal, Hyedrabad (IN)

(73) Assignee: NAGARJUNA ENERGY PRIVATE LIMITED, Punjagutta, Hyedrabad (IN)

(21) Appl. No.: 12/665,924

(22) PCT Filed: Jun. 19, 2008
(86) PCT No.: PCT/IB08/01602

§ 371 Date: Jul. 1, 2011

(30) Foreign Application Priority Data
Jun. 20, 2007 (IN) 2377/CHE/2006
Jun. 19, 2008 (IB) PCT/IB2008/001602

(51) Int. Cl. C12P 1/00; C12M 1/00

(52) U.S. Cl. 435/277; 435/267; 435/289.1; 435/303.1

(57) ABSTRACT

The present invention provides a continuous process for saccharification of cellulose by enzymatic degration without any loss of enzymes and also discloses a bioreactor for performing said process.

### FIELD OF INVENTION

The present invention is in the field of bio-chemical engineering.

### BACKGROUND AND PRIOR ART

Lignocellulosic biomass, a renewable source of energy derived from woody plants, agricultural residues, and other similar forms of biological matter. For effect of this invention, cellulosic and lignocellulosic materials are characterized as complex mixtures containing mainly cellulose, hemicellulose, lignin. Cellulose, which is a glucose polymer linked together by β, 1-4 linkages, present in proportions between 30% to 70% by weight depending on the type of lignocellulosic biomass.

The hydrolysis of cellulosic biomass by enzyme is a complex phenomenon affected by both the structure of the substrate and condition of reaction. However, to degrade such complex biomass it requires time and energy thereby increasing the process cost.

The enzyme cellulase, a biological catalyst, composed of several proteins, which is required to achieve rapid cellulose hydrolysis; however, use of this catalyst is not practical at present because it is very expensive and no satisfactory method so far been developed to recover the enzyme from the hydrolysate mixture for the purpose of reuse.

Additionally, the formed sugars tend to inhibit the catalytic action of enzymes thereby limiting the process to be industrially economic. While removing the sugar, some part or enzyme is also lost. These limitations allied to the high cost of enzymes available commercially render the processes of enzymatic hydrolysis not much attractive by an economic point of view.

U.S. Pat. No. 4,220,721 described method of cellulase reutilization from the SSF fermentation by separating the cellulose-cellulase complex after specified time and use the same as a an enzyme source for new SSF process after separating the product. U.S. Pat. No. 5,348,871 disclosed the process of continuous cellulose saccharification by two reactors wherein the first reactor having fixed bed for cellulose hydrolysis in presence of cellulase enzyme and the second rector contain cellobiose-hydrolyzing enzyme for hydrolysis of cellobiose into its monomeric products. U.S. Pat. No. 4,713,334 describes the process of enzymatic cellulose saccharification in aqueous media and separating the soluble saccharide and reutilizing the unhydrolyzed cellulose-cellulase complex for separate batch of saccharification. U.S. Pat. Nos. 5,258,293 and 5,837,506 show continuous reactor processes for saccharification and fermentation processes, and discuss a variety of reactor configurations. The enzymatic hydrolysis of cellulose could become a more economical process if the enzyme recovered from the reaction mixture in active form and reused several times. This can be achieved by immobilizing cellulase in some support to hydrolyze cellulosic substrates. However, the use of an immobilized enzyme to catalyze the hydrolysis of an insoluble substrate is difficult because effective interaction between enzyme and substrate would greatly impaired by the enzyme's immobility. There are, however, some reports describing the use of immobilized cellulase to hydrolyze insoluble cellulose.

In a nutshell, all the existing saccharification process suffers from following drawbacks:
a. Presence of more than 2% sugar in a saccharification process causes suppression of enzymatic activity. In order to maintain the sugar concentration below 2%, continuously sugar solution has to be removed from the system. During the removal process of sugar, cellulase enzyme is also lost, thereby rendering the process not economically viable.
b. During a continuous saccharification process, the un-reacted cellulose and impurities which are present along with starting material have to be constantly removed to allow the addition of new substrate. During this removal, enzyme is also lost causing in the increase the process cost.
c. Any subsequent process to recover the lost enzyme for reuse involves addition cost.

Because of these reason, saccharification process becomes expensive.

In order to overcome drawbacks as stated above, following conditions are to be fulfilled:
1. Enzyme should not be eluted along with the soluble products.
2. There should be means to remove impurities without loosing reactive cellulose and enzyme.

### OBJECTS OF THE PRESENT INVENTION

The main object of the present invention is to develop a saccharification process and a system to carry out the process where enzyme loss can be minimized or eliminated. One more object of the invention is to develop a continuous process having the above said advantages.

### DETAILED DESCRIPTION OF THE PRESENT INVENTION

Accordingly, the present invention discloses a process for hydrolyzing biomass by enzymatic degradation to produce its respective monomers, wherein the polymeric biomass is an insoluble solid and the enzyme is water-soluble and having a capacity to get adsorbed on the polymeric surface.

In one aspect, the present invention discloses a process and a reactor system developed for de-polymerization lignocellulosic biopolymers into its constituent fermentable monomeric sugars in continuous mode, wherein the enzyme loss is substantially eliminated.

In one aspect of the present invention, enzyme is made adsorbed on a substrate until enzyme saturation is attained to form enzyme-substrate complex and defined as first material. Only biomass, which is devoid of any enzyme herein after referred to as second material.

In one more aspect of the invention, a saccharification reactor is partially filled with the first material and remaining volume of the reactor is optionally packed with second material.

Further, in one more aspect, water is passed through the reactor in a predetermined rate to enable the cellulase to react with the substrate. Cellulase in a minute quantity keeps on moving upward due to two reasons. Firstly, along with the water movement, enzyme in very minute quantity moves upward and secondly, part of the enzyme after degrading the cellulose in the substrate also move upward along with flowing water and start reacting with un-reacted cellulose. During the ongoing degradation process, volume of first material starts shrinking and to compensate the same second material is added to the reactor system over the first material. Thus, enzyme is prevented from escaping along with water while collecting sugar solution, wherein the entire process maintained in such a way so that the rate of addition of second material is higher or equal than the rate of enzymatic hydrolysis.

The fortification of cellulase enzyme with cellobiase enzyme or incorporating the cellobiose enzyme at a later stage increased cellulose hydrolysis and raised the proportion of monomeric sugars in the hydrolysate. Continuous cellulose hydrolysis maintained for 80 to 100 hours at 30-70° C. preferably at 40-60° C. Greater than 87% (w/w) of the sugars produced were in the monomeric state.

In one more embodiment of the present invention, a reactor is designed to carry out the process of depolymerization of polymeric biomass.

### Description of the Reactor

Accordingly, the present invention provides an enzymatic bioreactor for hydrolyzing biomass. The enzymatic bioreactor of the present invention comprises an elongated chamber, preferably vertically placed. The elongated chamber has first region and second region. Preferably, the lower portion of the elongated chamber is a first region and the upper portion of the elongated chamber is a second region. The first region is a reaction chamber and provided with a first material saturated with one or more enzymes. The first material is biomass material saturated with enzymes. The second region is provided with a second material. The second material is pure biomass. The elongated chamber has one inlet at the bottom or near the first region for supplying water. An outlet is provided to the elongated chamber at the top or near second region for collecting the hydrolyzed material along with water. A second inlet can be provided to the elongated chamber for supplying second material or pure biomass. The first region is a reaction region; therefore, a predetermined temperature has to be maintained in the first region. To maintain the temperature water or steam jackets are provided to the elongated chamber.

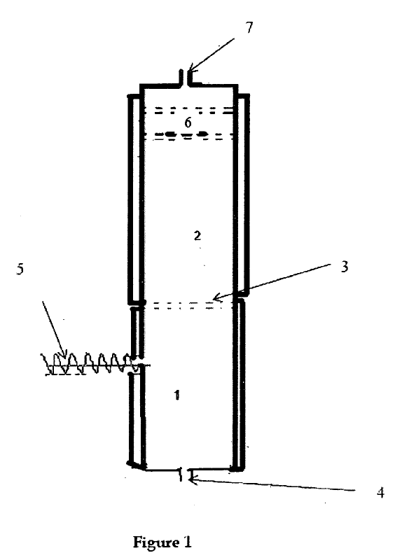

In a preferred embodiment of the present invention, the novel enzymatic bioreactor comprises of four chambers. As shown in FIG. 1, The bottom chamber (1) is the reaction chamber which is wall jacketed to control the reaction temperature inside the chamber at optimum condition and through the jacketed wall hot water or steam passed continuously through out the process to maintain the desired temperature inside the reaction chamber. The reaction chamber fitted on top a perforated plate (3) sufficient to stop the cellulose to pass the reaction chamber. Bottom of this chamber having an inlet (4) to feed the buffer at a desired flow rate. In one side of the chamber fitted with a screw type (5) feeder for inserting substrate from time to time. The second chamber (2) chamber packed with bagasse and the top of the chamber fitted with a fine mesh. The third chamber (6) filled with pellets of β-glucosidase enzyme immobilized in Na-alginate beads and top of the column covered with fine mesh which did not allow the pellets to go out. This portion of the chamber having an outlet facility (7). The entire chamber 2 and 3 jacketed outside to maintain desired temperature for β-glucosidase enzyme to saccharify the cellobioses, which produced in the bottom chamber 1.

The reaction chamber 1 filled with cellulose adsorbed cellulose enzyme and optionally added β-glucosidase enzyme. The temperature inside of the reaction chamber maintained at a temperature 30°-70° C. preferably 40°-60° C. by circulation of hot water though the jacketed wall and the temperature of the reaction chamber checked time to time with a digital thermometer. Feed particulate matter preferably cellulose fed through the screw type feeder, which positioned at the side of the reaction chamber. Buffered water whose pH adjusted to 3-6 more preferably 4-6 passed through the inlet situated at the bottom of the said reactor at a preferred flow rate, which is sufficient to maintain the process. After a predetermined holding time in the said reactor, the pH-adjusted water along with the product stream, which mainly contained cellooligosaccharide, preferably cellobiose, glucose and other un-dissociated sugars allowed to pass through the packed bed of area (2) of the column to the immobilized β-glucosidase pellet area (6). The entire area of the column 2 and 3 maintained at a temperature that is sufficient to breakdown the saccharides through the passage of hot water through the jacket. After a holding time in that particular area the entire liquid again circulated through the bottom inlet of first portion of reactor (1) until the sugar concentration of the outlet reached certain levels, which are not inhibitory to the enzyme. The feeder of the reactor chamber configured to receive the cellulosic biomass at a predetermined rate so that the adsorbed enzymes remain with the said solid matrix through out the process.

As the hydrolysis progress of the cellulosic substrate present in adsorbed form, the free enzyme is moving upwards but as the cellulose fed through the feeder, the available enzyme reacts with the incoming substrate and therefore enzyme remain virtually adsorbed through out process. Moreover, the packed bagasse bed above the reaction chamber pushing the free enzyme for effective saccharification. The entire process for hydrolysis depends on reaction rate, the flow rate and the substrate feed rate and are balanced such a way that the enzyme will remain in the bed.

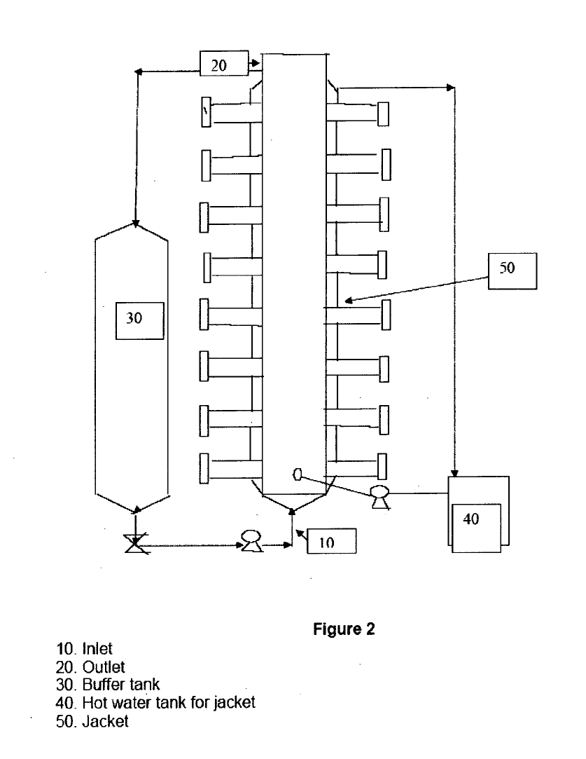

FIG. 2 illustrates the enzymatic bioreactor according another embodiment of the present invention. FIG. 2 depicts the reactor for the study of enzyme adsorption and reutilization of enzyme for continuous use. The reactor made with multiple parallel ports. The vertical distances between the ports are 5 cm and at the time of operation, ports were covered with dummies. At different time intervals, lignocellulosic samples collected from each port by pushing the sample from opposite side of the port. Person skilled in the art obviously understand the operating principle. The bottom of the reactor fitted with fine stainless steel mess to provide support to the lignocellulosic packed bed. Tap water whose pH adjusted between 3-7 passed through the inlet (10) situated at the bottom of the reactor with the help a pump and collected from outlet (20) positioned at the upper portion of the reactor. Both the inlet and outlet connected to the buffered water tank (30). The entire reactor jacketed from outside (50) and hot water (40) circulated in the same to maintain the internal temperature of the reactor between 30°-70° C. throughout the process.

The working of the saccharification process essentially comprising the steps of:
1. packing of the column reactor with the polymeric substrate to obtain a solid matrix
2. setting the buffer flow through the solid matrix by pump to initiate de-polymerization and subsequently remove product. The peristaltic pump was set to obtain a flow rate of 0.45 ml/min. About 100 ml of tap water pH adjusted to 4.5 was taken in a buffer tank capacity 250 ml. this was circulated from the inlet 1
3. eluting the buffer out through outlet and re-circulated into the column via buffer tank through inlet. This process continues until product concentration reaches the inhibitory level. When the inhibitory concentration reaches it is decanted and replaced with fresh buffer. As the cellulosic fibers are de polymerized the bed height, the feeder located at the upper part of the reaction chamber adds fresh substrate.

### EXAMPLES

The following examples are given by way of illustrations of the present invention and should not be considered to limit the scope of present invention.

### Example 1

90 gram of lignocellulosic material having approximately 65% cellulose and nearly 14% lignin mixed with approximately 450 FPU of commercial cellulase enzyme loaded into the reactor in a column having a dimension length 50 cm and diameter 3 cm, and fed into the reaction chamber 1. About 180 g, wet bagasse carefully packed in chamber 2 to make the packed bed. System runs for 0 to 96 hours. pH-adjusted water recycled at the rate of 50 μL per min. Eluting the sugar solution out through outlet 6 and re-circulated into the column via buffer tank through inlet 1. This process continues until product concentration reaches the inhibitory level. When the inhibitory concentration reaches it is decanted and replaced with fresh buffer. Due to shrinkage of bed-height, fresh substrate were added. From time to time eluted samples analyzed presence of protein. No protein was detected up to 96 hrs.

### Example 2

About 80 gms of ammonia and acid pretreated lignocellulosic sample containing approx 85% moisture mixed intimately with commercial enzyme preparation containing 423 FPU of cellulose and 280 CBU of cellobiase enzyme so the entire protein adsorbed into the substrate. The total mass of enzyme-substrate complex placed in the bottom portion of the reactor.

### Example 3

About 400 gms of pretreated lignocellulosic substrate loaded on top of the enzyme—substrate preparation as exemplified in Ex. 2 to completely load the reactor and made it a compact bed reactor. With reference to FIG. 2, pH adjusted water pumped from the bottom inlet (1) till the time when the eluted water reached a threshold of sugar concentration which is inhibitory to the enzyme. As the saccharification progresses fresh substrate continuously added from the top of the reactor to replenish the saccharified cellulose and to continue the process. Table 1 showed the result where saccharification almost reached nearly 87% whereas the lignin content is increased. In this process the enzyme, remain active till the process 96 hrs without additional enzyme loading and may be continued further as saccharification rate reached nearly 88%.

TABLE 1

| Time | bed height[cm] | lignin | cellulose | Saccharification |
|---|---|---|---|---|
| Hours | [cm] | [%] | [%] | [%] |
| 24 | 5 | 46.65 | 27.69 | 87.09 |
| | 10 | 34.55 | 44.49 | 71.99 |
| | 15 | 25.68 | 56.69 | 51.98 |
| | 20 | 20.01 | 46.19 | 49.78 |
| | 25 | 14.25 | 62.34 | 4.83 |
| | 30 | 14.54 | 64.32 | 3.77 |
| | 35 | 14.14 | 65 | 0 |
| 48 | 5 | 47.32 | 26.83 | 87.67 |
| | 10 | 36.14 | 40.44 | 75.66 |
| | 15 | 27.72 | 55.34 | 56.57 |
| | 20 | 21.74 | 48.21 | 51.76 |
| | 25 | 14.62 | 63.35 | 5.74 |
| | 30 | 14.48 | 64.24 | 3.49 |
| | 35 | 14.44 | 65 | 2.08 |
| 72 | 5 | 49.28 | 24.98 | 88.97 |
| | 10 | 36.98 | 38.29 | 77.48 |
| | 15 | 29.14 | 52.18 | 61.05 |
| | 20 | 25.16 | 43.69 | 62.22 |
| | 25 | 18.61 | 62.46 | 26.99 |
| | 30 | 14.62 | 64.19 | 4.49 |
| | 35 | 14.14 | 65 | 0 |
| 96 | 5 | 49.39 | 26.98 | 88.12 |
| | 10 | 34.74 | 38.64 | 75.8 |
| | 15 | 27.69 | 49.28 | 61.28 |
| | 20 | 23.55 | 40.32 | 62.76 |
| | 25 | 19.08 | 61.29 | 30.12 |
| | 30 | 14.69 | 64.19 | 4.94 |
| | 35 | 14.14 | 65 | 0 |

We claim:

1. A process of hydrolyzing biomass by enzymatic degradation, said process comprising the steps of:
a. providing a first material and optionally a second material in a system, wherein the first material is a water-insoluble solid biomass saturated with one or more enzymes capable of hydrolyzing said biomass and second material is only biomass;
b. passing water at a predetermined rate through the first and second materials thereby enabling the enzyme(s) to hydrolyze the biomass of the first material and leaving behind un-reacted residue;
c. collecting the hydrolyzed material along with water; and
d. removing the residue from the system, and compensating said removal with second material.
wherein the rate of addition of second material is equal or more than the rate of hydrolysis.

2. A process as claimed in claim 1, wherein the hydrolysis is carried out at a temperature in the range of 30 to 70° C.

3. A process as claimed in claim 1, wherein the biomass is cellulose.

4. A process as claimed in claim 1, wherein the enzyme is cellulase.

5. A bioreactor for hydrolyzing biomass by enzymatic degradation, said enzymatic bioreactor comprising:
An elongated chamber having a first region containing a first material saturated with one or more enzymes and a second region for containing a second material; said elongated chamber having a first inlet located near the first region for supplying water into the first region and an outlet near the second region for collecting the hydrolyzed material along with water.

6. A bioreactor as claimed in claim 5, wherein the elongated chamber is vertically placed so that the first region forms lower portion of the elongated chamber and the second regions forms upper portion of the elongated chamber.

7. A bioreactor as claimed in claims 5 and 6, wherein elongated chamber is provided with a metallic mesh dividing the elongated chamber into the first region and the second region.

8. A bioreactor as claimed in claims 5 to 7, wherein elongated chamber is provided with a second inlet for supplying the second material.

9. A bioreactor as claimed in claims 5 to 9, wherein the elongated chamber is provided with the means for maintaining temperature of the elongated chamber.
